(19) United States
(12) Patent Application Publication
(10) Pub. No.: US 2007/0081958 A1
(43) Pub. Date: Apr. 12, 2007

(54) Body care product containing porous silver particles

(76) Inventors: Thorsten Bechert, Forchheim (DE); Michael Wagener, Bremen (DE); Peter Steinrucke, Erlangen (DE)

Correspondence Address: PEARNE & GORDON LLP, 1801 EAST 9TH STREET, SUITE 1200, CLEVELAND, OH, 44114-3108, US

(21) Appl. No.: 10/570,230

(22) PCT Filed: Aug. 26, 2004
(86) PCT No.: PCT/EP04/09536

§ 371 Date: Aug. 17, 2006

(30) Foreign Application Priority Data
Aug. 29, 2003 (DE) 10340277.2

(51) Int. Cl. A61K 8/19; A61K 33/38

(52) U.S. Cl. 424/70.1; 424/618; 424/445; 977/926

(57) ABSTRACT

The invention relates to a body care product containing porous particles made of metal containing silver and having an average diameter size of between 1 and 100 μm.

The invention relates to a body care product and to a use for producing a medicament for treating an inflammation and/or infection.

WO 02/17984 A1 discloses an antimicrobial material for being implanted in bone or for coating or producing an implant or an implantable medical device. In the case of this material, particles which are formed from an antimicrobial metal are finely dispersed in a matrix material which, in the cured state, forms a matrix. The metal can be formed from one or more of the following constituents: Ag, Au, Pt, Pd, Ir, Sn, Cu, Sb, Zn.

WO 00/78281 A1 discloses an antimicrobial body care product which exhibits an organic matrix in a part which contacts human or animal skin and/or mucosa. This matrix contains homogeneously dispersed particles of metallic silver. In this case, the particles are between 1 and 50 nm in size. Particles of this size are what are termed nanoparticles. These particles are present in a quantity which, on the surface of the part which contacts the skin and/or mucosa, provides a concentration which is antimicrobially effective but less than cytotoxic. The body care product can, for example, be an ointment or a cream.

It is known from Brumfiel, G. Nature (2003), Vol. 424, pages 246 to 248, that nanoparticles can be taken up by animals. For example, nanoparticles can pass from the lung into the bloodstream. It is not yet clear what effects nanoparticles have on human health when they have penetrated into the body. The effects on human health of the nanoparticles which are present in the body care product in accordance with WO 00/78281 A1, and which are composed of silver, are therefore not clear, either.

DE 693 21 139 T2 discloses an antimicrobial composition which is composed of inorganic particles which can be coated with metallic silver. The particles can be incorporated in a polymer. They have a diameter of from 0.01 to 100 μm, i.e. they can also be present in the form of nanoparticles. The object underlying DE 693 21 139 T2 is to provide antimicrobial particles which can be readily incorporated into a polymer matrix in connection with which interaction with the polymer is minimized. The particles possess a protective layer of low porosity on top of the silver and are readily dispersible in the polymer. The protective layer is intended to prevent the metal from coming into too intensive a contact with its environment.

DE 38 86 193 T2 discloses a titanium-mica composite material which can be used as a pigment in cosmetics and which possesses a coating composed of metallic pulverulent silver. The object underlying DE 38 86 193 T2 is to provide a material which is suitable for being used as a dye or pigment.

WO 00/78282 A1 discloses a silicone rubber compound which contains metallic silver particles of from 1 to 50 nm in size. The silver particles are present in a quantity which provides, on the surface of said compound, a concentration of silver which is antimicrobially effective but less than cytotoxic.

JP 61257908 A discloses a make-up composition which comprises a power which is coated with a metal powder, e.g. composed of silver. In this case, the silver coating evidently only serves the purpose of providing optical properties.

The object of the present invention is to provide a body care product which possesses antimicrobial activity and which does not exhibit the nanoparticle-associated disadvantages of the body care product disclosed in WO 00/78281 A1. Furthermore, a use for producing a medicament for treating an inflammation in a mammal or human is to be specified.

The object of the present invention is achieved by the features of patent claims 1 and 16. Expedient embodiments ensue from the features of claims 2 to 15 and 17 to 31.

The invention provides for a body care product which comprises porous particles which contain metallic silver, which are formed from metal and which have a mean diameter of between 1 and 100 μm.

Body care products are products which are brought into contact with the human or animal skin and/or mucosa in order to achieve a cleaning, protective, therapeutic, healing, caring, cosmetic or alleviating effect. For example, these are products which usually exhibit surfaces which contact the skin and are composed of a natural or synthetic polymer material. These products can, for example, be absorbent disposable articles such as feminine hygiene articles, in particular sanitary napkins, panty liners or tampons, incontinence liners, diapers, baby training pants, medical bandages, plasters, nonwoven materials, textiles, cellulose, toothbrushes or pacifiers. The body care products can be produced from a natural product such as wool, viscose, cellulose and derivatives thereof or natural rubber, or comprise these natural products. They can also be produced from plastics or comprise plastics which contain the porous particles which contain metallic silver. The plastics can, for example, be: polyethylenes and copolymers derived therefrom, polypropylenes and polyblends prepared therefrom, polybutenes, polystyrenes in homopolymers and copolymers, acrylic-butadiene-styrene-terpolymer (ABS), synthetic rubbers, hard and soft PVC, polytetrafluoroethene (PTFE), polychlorotrifluoroethylene (PCTFE) and other fluoropolymers, polyvinyl ethers, polyvinyl acetates, polyvinyl propionates, polyvinyl alcohols, copolymers of vinyl alcohol, polyvinyl acetals, polyethylene glycols, acrylic polymers, polymethacrylic acid methyl ester, polyacrylonitrile, polycyanoacrylates, polymers based on polymethacrylimide, polyacrylimides, polyvinylamines, polyamides including polyphenyleneisophthalamide, poly(p-phenyleneterephthalamide), linear polyurethanes and polyesters including polyethyleneterephthalate (PET), polybutyleneterephthalate (PBT) and polytetramethyleneterephthalate (PTMT), polycarbonates and polymers derived therefrom, polyoxymethylenes (POMs), polyethers, polyether ether ketones, polyether block amides, condensation resins, such as phenoplasts and aminoplasts, crosslinked polyesters including polyester resins, epoxy resins, crosslinked polyurethanes, reaction resins based on methyl methacrylate, polysiloxanes and other polymers having an inorganic main chain.

The body care products can also be preparations which are, in particular, medicinally active, such as emulsions, lotions, gels, creams, ointments, healing ointments, powders, cosmetics, skin protection creams or ointments, disinfectants or antiinflammatory remedies, suspensions, soaps, synthetic surfactants, bath additives, peeling preparations, face lotions, tooth care products, toothpastes, mouthwashes, tooth-cleaning chewing gums, denture adhesives, hair shampoos, sunscreen agents, etc. These products frequently contain either a polymer or an organic constituent in a carrier which can be a good substrate for a large number of microorganisms. Growth of these microorganisms in these substrates can give rise to hygienic or medical problems.

The particles can be present in the body care product in a quantity which enables a concentration of silver ions, which is microbially effective but less than cytotoxic, to be present at a site at which the body care product makes contact with the skin and/or mucosa.

Because of their size being from 1 to 100 μm, the particles which are present in the body care product according to the invention and which are composed of metal do not pose the potential risks of nanoparticles. When the body care product is employed in accordance with its intended use, the particles are unable to penetrate through the deeper skin layers into tissue or into blood vessels and nor can they overcome the blood-brain barrier, either. As a result, the antimicrobial effect is restricted solely to the skin surface. This thereby avoids inducing allergies and undesirable toxic effects. It has been found, however, that the silver ions, which can be released from the particles due to their porosity, are sufficient to be able to provide a body care product which possesses antimicrobial and, where appropriate, antiinflammatory activity. An antiinflammatory effect can be achieved when the particles are present in the body care product at a concentration which is higher than that which is required for achieving what is merely an antimicrobial effect. The silver ions act, in particular, on the surface of the skin or mucosa which makes contact with the body care product and have no negative influence on underlying tissue. As a result, and because of their size which prevents penetration into the skin, the particles are very much more skin-compatible than are nanoparticles. The particles are less cell-damaging and more biocompatible than are nanoparticles which contain metallic silver. As a result, the body care product according to the invention is suitable, in particular, for patients who have to pay increased attention to body care and body hygiene over the long term. These patients can, for example, be individuals, such as diabetics, who have a weakened immune system and/or run an increased risk of contracting skin infections. Since it has been found that the body care product according to the invention frequently renders the additional use of antibiotics superfluous, it is thereby also possible to prevent the development of antibiotic resistances.

The body care product according to the invention has an antimicrobial effect and, where appropriate, an anti-inflammatory effect at the same time. In addition to this, because of the antimicrobial effect of the metallic silver, the product does not require any preservatives in addition to the particles. It can be present in the form of, in particular, a medical healing or caring ointment, cream or gel. Because of the antiinflammatory effect, such a preparation can be used medically as an alternative to corticoid-containing preparations. When the product is used as a hand ointment, cream or gel, the antimicrobial effect also protects against the transfer of pathogens, e.g. by means of handshaking, and prevents organism penetration when there are small wounds on the hands. In addition to this, because preservatives can be dispensed with, fewer incompatibility, in particular allergic, reactions occur.

The particles preferably have a mean internal porosity of at least 65%, in particular of between 65 and 95%. Internal porosity is understood as meaning the percentage of the volume of the particle which is not filled with metal. The mean internal porosity of the particles can be determined using the following method:
1. embedding the particles in a plastic,
2. preparing ultrathin sections of the embedded particles,
3. taking transmission electron microscopic (TEM) photographs of the particles,
4. determining the percentage of the area within each particle which is not filled with metal, in relation to the total area of this particle, in a plurality of TEM photographs, and
5. calculating the mean of a plurality of percentages which have been determined in this way.

In this connection, step 4 can be effected by means of a computer-assisted image analysis of the TEM photographs. In addition to the internal porosity, it is also possible to determine the total porosity of the particles. For this, the beating density of a powder of the particles is first of all determined. The beating density is the mass of a unit volume of a powder which is layered as densely as possible by means of beating. The beating density can be determined in accordance with DIN ISO 3953. The value which is determined in this connection is calculated as a percentage of the density of the metal forming the particles, in this case silver having a density of 10.49 g/cm³, and subtracted from 100%. The value which is calculated in this way constitutes the total porosity of the particles. In the case of the particles which are contained in the body care product according to the invention, it can be between 85 and 95%, in particular between 90 and 95%, preferably between 93 and 95%.

It is particularly advantageous for the particles to have a mean internal porosity of between 65 and 90%, in particular of between 70 and 85%, preferably of between 75 and 85% or of between 85 and 95%, preferably of between 90 and 95%. The choice of the porosity can be used to specify the quantity of silver ions which are released by a particle in a particular unit of time. If a high porosity is chosen, this thereby releases many silver ions which means that, overall, the antimicrobial and antiinflammatory effect is achieved using a lower quantity of silver in the body care product. On the other hand, the total duration of the release of silver ions is reduced by increasing the porosity and at the same time reducing the quantity of silver. A porosity of between 70 and 85%, or between 85 and 95%, is therefore advantageous, depending on the application.

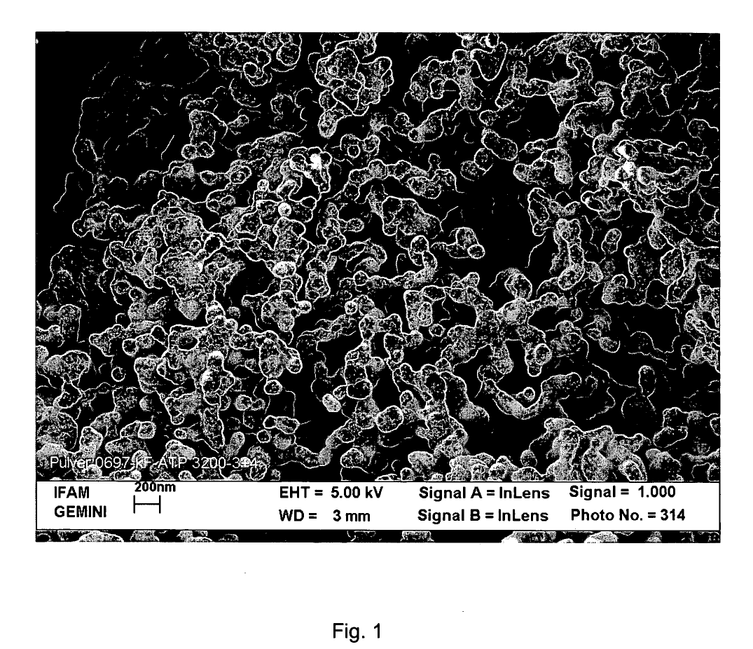

The particles are preferably present as agglomerates of metallic primary particles. The agglomerates can be formed from primary particles having a mean diameter of between 10 and 200 nm, preferably of between 15 and 80 nm. Primary particles of this size permit adequate release of silver ions and can be readily produced. The mean distance between the in each case outermost primary particles at the surface of the agglomerates is preferably in the range of from 20 to 200 nm, preferably of from 100 to 200 nm. The primary particles can be identified electron microscopically on the basis of their external shape and size. They can be seen, for example, as spherical structures in FIG. 1. The primary particles are connected to each other by way of sinter necks.

The porous particles preferably have a sponge-like structure. The large surface which this thereby provides makes it possible to release silver ions in adequate quantity for them to have an antimicrobial and, where appropriate, antiinflammatory effect.

The particles preferably have a mean external diameter of from 2 to 20 μm, preferably of from 2 to 5 μm. The specific surface of the particles can be between 2 and 10 m²/g, in particular between 3 and 6 m²/g, preferably between 3.5 and 4.5 m²/g. The specific surface can, for example, be determined volumetrically by means of N₂ adsorption using the BET method. The BET method is a method, which is named for Brunauer, Emmett and Teller, for determining the surface and, where appropriate, the pore size distribution as well, of solid bodies (e.g. powders), with the method being based on gases, vapors, etc. being initially adsorbed in a monomolecular layer on solid bodies with the release of a measurable heat of adsorption. For example, it is possible to measure the volume of nitrogen gas which is adsorbed on the adsorbent at −196° C. in dependence on the pressure which is applied.

The particles preferably consist of at least 99% w/w (percent by weight), preferably 99.9% w/w, metallic silver. At such a high silver content, there is no noticeable cytotoxic effect due to other metal ions, in particular copper ions. Unless indicated otherwise, the percentage metal contents which are given here and in that which follows refer to the weight of the given metals as a percentage of the total weight of the particles. This is expressed as percentages by weight (% w/w). It is particularly advantageous if the particles comprise less than 5 ppm of potassium, sodium or chlorine impurities. Higher quantities of impurities in the silver can give rise to undesirable side effects.

It is particularly advantageous if the particles comprise up to 0.5% w/w metallic zinc and/or up to 0.5% w/w metallic copper. Both substances also have an antimicrobial effect and support each other mutually, together with the silver, in their effects. This is due, inter alia, to the fact that, in their antimicrobial effects, they exhibit different specificities for microorganisms. In addition, zinc exhibits a particularly good wound-healing and antiinflammatory effect in combination with silver and, where appropriate, copper. The reason for this could be that the silver and the copper, which is present where appropriate, prevent the growth of microorganisms which interfere with the wound healing and whose growth is not inhibited by zinc ions on their own. In addition to this, the copper facilitates the production of an alloy composed of zinc and silver. Taken overall, the body care product which comprises zinc and/or copper in addition to silver has a better wound-healing and antiinflammatory effect than a body care product which in each case comprises only one of the metals. Preference is given to the particles being formed from a silver-zinc alloy or a silver-zinc-copper alloy.

The body care product preferably does not comprise any preservatives in addition to the particles. It has been found that the metal ions exhibit a preservative effect. It is therefore possible to dispense with preservatives. This thereby makes it possible to avoid undesirable, in particular allergic, reactions which are induced by a customary preservative such as formaldehyde.

The particles can be present in a carrier material which consists of a silicone oil, a mineral oil, glycerol or a customary ointment constituent which is known from pharmacology. In order to produce a body care product according to the invention, the agglomerates can be produced by thermally evaporating the agglomerate-forming metal and then depositing the metal vapor on a metal filter. The agglomerates can be taken up in a carrier material which is introduced into the body care product. The carrier material can, for example, be a silicone oil, a mineral oil, glycerol or a customary ointment constituent which is known from pharmacology.

In addition, the invention relates to the use of porous particles which contain metallic silver, which are formed from metal and which have a mean diameter of between 1 and 100 μm for producing a medicament for treating an inflammation and/or infection in a mammal or human. Customary medicaments for treating an inflammation in a mammal or human frequently comprise a combination of antiinflammatory and antimicrobial active compounds. The antimicrobial active compound is intended to prevent or control an infection, in particular with Staphylococcus aureus. The antimicrobial active compound is usually an antibiotic. Alternatively, the antibiotic can also be administered systemically while the antiinflammatory active compound is administered locally, e.g. topically. However, because of the danger which exists, in particular in connection with long-term use, of antibiotic resistances developing, the use of antibiotics should be reduced to a minimum. An example of an antiinflammatory active compound which has previously been used is a corticoid such as cortisone which, however, displays a large number of side effects. The essential advantage of the medicament which is produced in accordance with the invention is that the particles display both an antiinflammatory effect and an antimicrobial effect. The use of antibiotics can be reduced and the side effects of the corticoids or other antiinflammatory active compounds can be avoided.

The treatment preferably takes place topically, i.e. by, for example, application to the skin or a wound. The medicament can be an ointment, a cream or a gel. Other advantageous embodiments of the use ensue from the above comments concerning the body care product according to the invention.

The invention is explained in more detail below with the aid of exemplary embodiments. With regard to the figures:

FIG. 1 shows a scanning electron microscopic photograph of a silver agglomerate, and

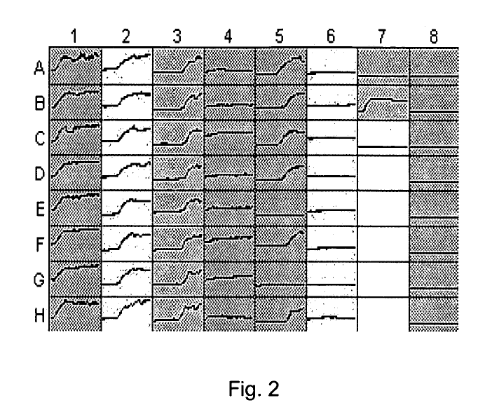

FIG. 2 shows a matrix of plots of the chronological course of the growth, which is measured in the form of the optical density (OD) of a medium, of bacteria which are in contact with different creamy body care products.

FIG. 1 shows a scanning electron microscopic photograph of a silver agglomerate. In this case, the silver agglomerate essentially comprises spherical primary particles having a mean particle size of about 60 nm. The primary particles are essentially connected to each other by way of sinter necks. They form a highly porous framework. The size of the silver agglomerate which is shown here is about 10 μm.

The results which are shown in FIG. 2 have been determined using the method which is disclosed in DE 197 51 581 A1. This method is also described in Bechert, Thorsten et al., Nature Medicine (2000), Vol. 6, No. 8, pages 1053 to 1056. The disclosure content of the two abovementioned documents is hereby incorporated herein by reference. The body care products according to the invention which were to be tested were produced in the form of creams, in each case applied to a material acting as carrier and then used in the test as described. In detail, the test was carried out as follows:

Different cream samples are produced first of all. A quantity of 11 mg of the given cream is applied to each carrier. 200 μl of a solution containing Staphylococcus epidermidis are then aliquoted into each well in a microtiter plate. The carriers together with the cream samples are in each case incubated in one of the wells at 37° C. for one hour. The carriers are then removed and washed three times with physiological buffer. The carriers are then in each case laid in a microtiter plate well which contains 200 μl of a minimal medium. The carriers are incubated at 37° C. for 24 hours. The carriers are then removed and discarded. 50 μl of a complete medium (Trypcase-soya, bioMerieux, No. 69280, Marcy l'Etoile, France) are added to each well in the microtiter plate. The turbidity of the solution is then measured at intervals of 30 minutes over a period of 48 hours. During this period, the solution is kept at a temperature of 37° C. The turbidity is measured with light at a wavelength of 578 nm using a suitable reading appliance. Turbidity indicates that bacteria have been released from the surface of the carrier into the environment.

“Cremaba Plus HT” from Spinnrad®, Certus Handels GmbH, 22848 Norderstedt, Germany was used as the basal cream for producing the cream samples. This basal cream is an emulsion base having the following ingredients: water, caprylic/capric triglycerides, pentylene glycol, hydrogenated lecithin, butyrospermum Parkii, glycerol, squalane and ceramide 3. The following additional constituent was incorporated into the basal cream:

silicone oil having a silver content of 0.65% w/w; in the oil, the silver is in the form of particles having a mean diameter of 10 nm; the silver is described below as being “nanodisperse silver”; or

agglomerates of metallic silver which are present in powder form and which have a mean porosity of 80% and a mean diameter of 5 μm; the silver is described below as being “agglomerate silver”.

Creams comprising 0.01% w/w nanodisperse silver and comprising 0.1% w/w and 0.5% w/w agglomerate silver were prepared. In addition, a cream comprising 0.05% w/w nanodisperse silver was prepared, with the nano-disperse silver in this case consisting of an alloy which consisted of 99.5% w/w silver, 0.49% w/w zinc and 0.01% w/w copper. In addition, a cream comprising 1.5% w/w agglomerate silver was prepared, with the agglomerate silver in this case consisting of an alloy which consisted of 99.5% w/w silver, 0.49% w/w zinc and 0.01% w/w copper.

In order to prepare the creams, the substances were in each case mixed in a 50 ml glass beaker and heated at 75° C for 20 minutes in a water bath; they were then dispersed for 5 minutes using an Ultraturrax (Janke and Kunkel, T25 drive, stator diameter 25 mm, rotor diameter 17 mm). The cream was then cooled and thoroughly mixed once again.

FIG. 2 shows each field of an x-y graph in which the time is plotted on the x axis and the optical density is plotted on the y axis. The experimental results depicted in columns 1 to 8 in FIG. 2 were determined, using the following creams, in parallel experimental assays A to H corresponding to the rows A to H:
column 1, rows A-H: cream without any additions of silver
column 2, rows A-H: cream comprising 0.1% w/w agglomerate silver
column 3, rows A-H: cream comprising 0.5% w/w agglomerate silver
column 4, rows A-H: cream comprising 1.5% w/w agglomerate silver consisting of 99.5% w/w silver, 0.49% w/w zinc and 0.01% w/w copper
column 5, rows A-H: cream comprising 0.1% w/w nanodisperse silver
column 6, rows A-H: cream comprising 0.05% w/w nanodisperse silver consisting of 99.5% w/w silver, 0.49% w/w zinc and 0.01% w/w copper
column 7, row A: positive control
column 7, row B: negative control
column 7, row C: blank value
column 8, rows A-H: sterile controls

A polymer containing metallic silver was used in the case of the positive control. The values show that the bacteria employed are sensitive to silver and can be killed by it. The same polymer was used in the case of the negative control but did not contain any silver. The blank value is a value which was measured in an empty well in the microtiter plate and which was subtracted when analyzing all measured values. In each case only medium, without any addition of Staphylococcus epidermidis, was used in the case of the sterile controls in order to demonstrate that the bacterial growth does not derive from the medium.

The results can be summarized as follows:

| | | Onset OD | |
|---|---|---|---|
| | Onset OD [h] | [h] | |
| Sample designation | gross | net | Effect |
| 1A-H cream without | 5.2 | 0 | not antibacterial |
| any additions of | | | |
| silver | | | |
| 2A-H cream comprising | 18.4 | 13.2 | highly |
| 0.1% w/w | | | antibacterial |
| agglomerate silver | | | |
| 3A-H cream comprising | 32.2 | 27.0 | highly |
| 0.5% w/w | | | antibacterial |
| agglomerate silver | | | |
| 4A-H cream comprising | 37.9 | 32.7 | highly |
| 1.5% w/w | | | antibacterial |
| agglomerate silver | | | |
| consisting of 99.5% | | | |
| w/w silver, 0.49% | | | |
| w/w zinc and 0.01% | | | |
| w/w copper | | | |
| 5A-H cream comprising | 35.3 | 30.1 | highly |
| 0.01% w/w | | | antibacterial |
| nanodisperse silver | | | |
| 6A-H cream comprising | limit | >42.8 | bactericidal |
| 0.05% w/w | | | |
| nanodisperse silver | | | |
| consisting of 99.5% | | | |
| w/w silver, 0.49% | | | |
| w/w zinc and 0.01% | | | |
| w/w copper | | | |
| 7A/B positive | limit/9.2 | — | OK |
| control/negative | | | |
| control | | | |
| 8A-H sterile | limit | — | OK |
| controls | | | |
| 7 C blank value | | — | OK |

“Onset OD [h] gross” denotes the time, measured in hours, until there was an exponential increase in the optical density (OD) by 0.2. “Onset OD [h] net” is obtained from “Onset OD [h] gross” by in each case subtracting the “Onset OD [h] gross” value which was determined for the cream to which no silver was added. Where parallel experimental assays were carried out, the mean value is given in each case. “Antibacterial” denotes an effect in which the growth of the bacteria is delayed while “bactericidal”. denotes an effect in which 100% of the bacteria are killed such that no bacterial growth can any longer be observed.

The experimental results show that agglomerate silver, like nanodisperse silver, has high antibacterial activity. Nanodisperse silver is active at lower silver concentrations than agglomerate silver. However, a highly antibacterial effect can still be achieved when using agglomerate silver. Both the effect of the agglomerate silver and the effect of the nanodisperse silver are increased in the creams which also comprise zinc and copper in addition to silver.

1-31. (canceled)

32. A body care product for application to skin and/or mucosa containing porous particles which are formed of metal and which contain metallic silver, said particles having a mean diameter of between from about 1 micron to about 100 microns.

33. The body care product of claim 32, wherein the particles have a mean internal porosity in the range of from about 65% to about 95%.

34. The body care product of claim 33, wherein the particles are present as agglomerates of metallic primary particles.

35. The body care product of claim 34, wherein the primary particles have a mean diameter of between from about 10 nm to about 200 nm.

36. The body care product of claim 35, wherein the mean distance between outermost primary particles at a surface of the agglomerates is in the range of from about 20 nm to about 200 nm.

37. The body care product of claim 33, wherein the particles have a sponge-like structure.

38. The body care product of claim 33, wherein the particles have mean external diameter of from about 2 microns to about 20 microns.

39. The body care product of claim 33, wherein the particles have a specific surface of from about 2 m²/g to about 10 m²/g.

40. The body care product of claim 33, wherein the total weight of the particles is at least 99% metallic silver.

41. The body care product of claim 40, wherein the particles comprise less then about 5 ppm of potassium, sodium or chlorine impurities.

42. The body care product of claim 40, wherein the total weight of the particles is up to about 0.5% metallic zinc and/or up to about 0.5% metallic copper.

43. The body care product of claim 40, wherein the particles are formed from a silver-zinc alloy or a silver-zinc-copper alloy.

44. The body care product of claim 33, wherein the body care product does not comprise any preservatives in addition to the particles.

45. The body care product of claim 33, wherein the particles are present in a carrier material selected from the group consisting of a silicone oil, a mineral oil, glycerol or an ointment constituent.

46. The body care product of claim 33, wherein the body care product is a preparation selected from the group consisting of an emulsion, a lotion, a gel, a cream, an ointment, a healing ointment, a powder, a cosmetic, a skin protection cream or ointment, a disinfectant, a suspension, a soap, a synthetic surfactant, a bath additive, a peeling preparation, a face lotion, a tooth care product, a toothpaste, a mouthwash, a hair shampoo or a sun-screen agent.

47. The body care product of claim 32, wherein the particles have a mean internal porosity in the range of from about 85% to about 95%.

48. The body care product of claim 47, wherein the particles are present as agglomerates of metallic primary particles and the primary particles have a mean diameter of between from about 15 nm to about 80 nm.

49. The body care product of claim 48, wherein the mean distance between outermost primary particles at a surface of the agglomerates is in the range of from about 100 nm to about 200 nm.

50. The body care product of claim 49, wherein the particles have a specific surface of from about 3.5 m²/g to about 4.5 m²/g.

51. A method for treating an inflammation and/or an infection, comprising applying to skin and or mucosa of a mammal or human having the inflammation and/or infection a medicament including porous particles which are formed of metal and which contain metallic silver, said particles having a mean diameter of between from about 1 micron to about 100 microns.

52. The method of claim 51, wherein the particles have an internal porosity in the range of from about 65% to about 95%.

53. The method of claim 52, wherein the particles are present as agglomerates of metallic primary particles.

54. The method of claim 53, wherein the primary particles have a mean diameter of from about 10 nm to about 200 nm.

55. The method of claim 54, wherein the mean distance between outermost primary particles at a surface of the agglomerates is in the range of from about 20 nm to about 200 nm.

56. The method of claim 52, wherein the particles have a sponge-like structure.

57. The method of claim 52, wherein the particles have a mean external diameter of from about 2 microns to about 20 microns.

58. The method of claim 52, wherein the particles have a specific surface of from about 2 m²/g to about 10 m²/g.

59. The method of claim 52, wherein the total weight of the particles is at least 99% metallic silver.

60. The method of claim 52, wherein the particles comprise less than about 5 ppm of potassium, sodium or chlorine in impurities.

61. The method of claim 52, wherein the total weight of the particles is up to about 0.5% metallic zinc and/or up to about 0.5% metallic copper.

62. The method of claim 52, wherein the particles are formed from a silver-zinc alloy or a silver-zinc-copper alloy.

63. The method of claim 52, wherein the medicament does not contain any preservatives in addition to the particles.

64. The method of claim 52, wherein the treatment is a topical treatment.

65. The method of claim 52, wherein the medicament is an ointment, a cream or a gel.

66. The method of claim 52, wherein the particles are present in the medicament in a carrier material selected from the group consisting of a silicone oil, a mineral oil, glycerol or an ointment constituent.

67. The method of claim 51, wherein the particles have a mean internal porosity in the range of from about 85% to about 95%.

68. The method of claim 67, wherein the particles are present as agglomerates of metallic primary particles and the primary particles have a mean diameter of between from about 16 nm to about 80 nm.

69. The method of claim 68, wherein the mean distance between outermost primary particles at a surface of the agglomerates is in the range of from about 100 nm to about 200 nm.

70. The method of claim 69, wherein the particles have a specific surface of from about 3.5 m²/g to about 4.5 m²/g.
